(19) United States
(12) Patent Application Publication
(10) Pub. No.: US 2021/0358590 A1
(43) Pub. Date: Nov. 18, 2021

(54) Method for reducing the harmful effects of physical inactivity, for initiating activity, and respective device

(71) Applicant: György Vasvári, Budapest (HU)

(72) Inventor: György Vasvári, Budapest (HU)

(21) Appl. No.: 17/285,136

(22) PCT Filed: Oct. 15, 2019
(86) PCT No.: PCT/HU2019/000031

§ 371 Date: Apr. 14, 2021

(30) Foreign Application Priority Data
Oct. 17, 2018 (HU) P1800354

(51) Int. Cl. G16H 20/30; G16H 40/67; G16H 40/20; G01L 5/00; A63B 24/00; A63B 71/06; A61G 5/10; A63B 21/055

(52) U.S. Cl. CPC G16H 20/30; G16H 40/67; G16H 40/20; G01L 5/00; A63B 2022/0094; A63B 24/0087; A63B 71/0622; A61G 5/10; A63B 21/0552; A63B 24/0062

(57) ABSTRACT

The problem of inactivity can occur at any area of our lives. Inactive periods can originate from being at hospital, rehabilitation, from physical condition, even during daily routine, or at leisure time. Several medical research discuses the irreparable harm of regular inactivity. The method and respective device according to the invention urges the person from harmful inactivity to activity, by signaling and measuring device, or by flexible displacing device. The signaling and measuring device or the flexible displacing device are coupled onto objects used in our daily life, thus activity can happen in our daily routine, at places where we at regularly. Activity happens by applying muscle force with controlled size, length of time and number of repetitions. Signaling and measuring device or flexible displacing device according to the method are coupled to other objects, such as bed or table. Signal initiates activity, sensors measure the engagement of proper muscle force and the signal is turned off accordingly. Similarly, flexible displacing device coupled to other objects is used to displace the inactive person in a controlled manner, by applying flexible force with controlled size over controlled length of time in retainable, stoppable form. The method and respective devices can be implemented at many possible circumstances, for example in healthcare institutions.

The subject of the invention is such method and respective device, by which the negative effects of the lengthy periods of physical inactivity can be decreased. The ill effects of physical inactivity to our health are part of our common knowledge. Such periods of physical inactivity for example are the time spent on sick beds at hospitals, at rehabilitation centers, at elderly care centers and at home, being in wheelchair, having certain disability or elderly condition, in many cases being at work doing the work, or during commute and travel. Furthermore, long periods are spent as well, in physical inactivity during our leisure time, in front of the PC, TV, Internet, during reading, or music listening. Actually inactivity can occur at any body positions. Obvious body positions are laying and sitting. But at todays world we can be inactive at all body positions, even standing, leaning or holding onto something, it all depends on the available circumstances and constraints effecting our actions. By the result of the invention the inactive person will begin activity, muscle work will be initiated, the joints will have movement, internal organ functions will improve. It will also have positive effect on overall bodyweight, looks and self-satisfaction.

Since the human personality usually selects the easier path and in most cases comfort overcomes self-discipline, to reach physical activity from inactivity for no certain reason, special method and device is necessary. The goal of this invention is such method and device, which has the effect to forward the person from inactivity into activity, at such circumstances where the place and possibility of activity is not obvious, perhaps only its necessity.

At the current stage of technical advancement such method or device has not been developed, therefore it is hard to make reference to previous inventions or solutions from the past. About the problems of inactivity many ideas and research has been put forward, but solutions that can be applied in the daily life have not been proposed. Anyhow, somewhat similar direction the lifting function of hospital beds, or the under desk bicycles, or the line of body-exercises that can be done on planes, in sickbeds. Such method that support usable solutions, devices, that are actually people are widely using, are not available.

During our physically inactive periods, we are many times surrounded by others, moreover, we are in such an environment where we have obligations regarding our behavior, such as hospital, workplace, family, travel, or the like. It is especially important to supply such method, device, which minimally annoy the environment, do not cause disturbance, and fits into the place's atmosphere. Other key factor, the presence of urge to begin activity, to do the necessary movement, because only the most self-discipline people are able to do regular exercises on their own.

### DESCRIPTION OF INVENTION

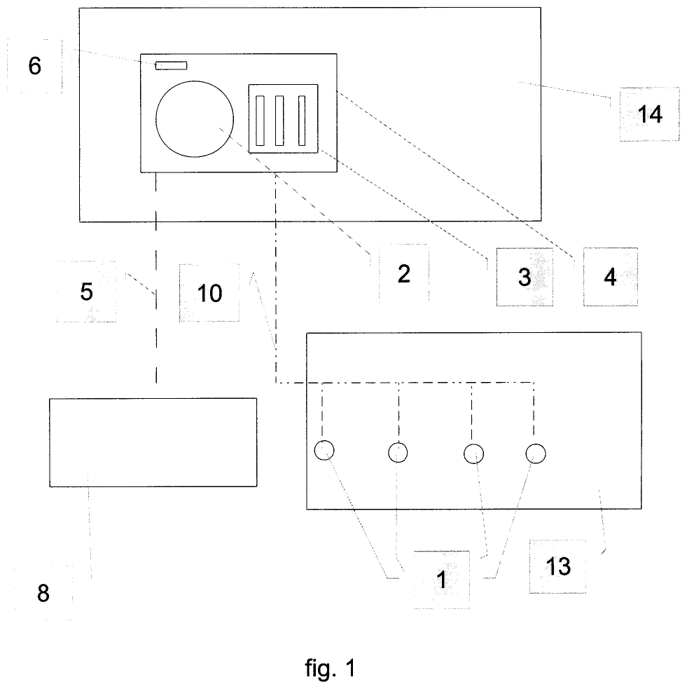

Method for initiating physical activity and suitable device could have one of the preferred embodiment as follows; Signaling and measuring device comprising of signaling equipment with data transmission capable connection (5, 10) to force measuring sensor (1). The signaling equipment is further comprising of signal source and controller component (8). The signal has the from of light-source (2), sound-source (3), or other available sources. The signaling equipment functions digitally, it is programmable, its function is to control and initiate the activity and to process the data from the sensors. In case of hospital bed (13) (FIG. 1) the sensors (1) are located integrally on the bed structure (13), at such places where they are accessible by hands or feet. The signaling equipment is coupled to the nurse call-system console (14), where the necessary electricity requirement is available, and it has wire connection (10) to the sensors (1). The signaling equipment regulates the signal in a controlled manner and processes the measured values of forces and durations received from the sensors (1). It offers the selection between different preprogrammed exercise routines. Selection can be made through wireless connection (5) by in-house controller (8) device. The method of exercise is the following; nurse turns on the signaling equipment coupled to call-system console (4) by on/off switch (6) and selects exercise routine using the controller (8). To the urge of light signal (2), force has to be applied onto predetermined points with controlled dimension, direction and duration as long as the signal suggests otherwise, for example by turning off. If measured values received from the sensors (1) are insufficient, then additional sound signal (3) activates. The repetition of the exercise, if there is any, the required level and duration of the forces acting on the sensors (1), and the selection of sensors (1) participating are all controlled by the signaling equipment coupled to call-system console (4). The size and duration of the applied forces are measured on prepositioned sensors (1). Final turning off is automatic. Furthermore, the sensors (1) or the signaling equipment can even be freely relocatable versions, in wired or wireless from. In case of the hospital bed (13), the signal can be light, sound, visual on screen, vibration, or even in the form that touches the body and felt through the skin. The sensors (1) or the signaling equipment can as well be installed to any objects in use, such as table, bed, chair, wall, cabinet, or the like, or to any other element such as to a draw, keyboard holder, or the like.

The type of the signal can be simple or complex. Can be simple point of light, but it can be numeric also, even showing force and time values. It can be simple sound, but it can be human sound forming as well. It can be vibrating, but it can be the form of touch on specific location on the human body. The urging effect of the signal is as efficient, as well it is capable to motivate the person to do the exercises. Such solution could be the level of sound, which can even bother the environment. It could be such visual on screen, that could block out previous information. It could be such illumination that can effect the view. It could be such vibration or touching that could cause uncomfortable feeling. In case of more than one person in a location, it would be logical to apply the physical activity simultaneously for the least botheration. In case of simultaneity the intensity of work can be increased, hence all participants are busy doing their own exercises.

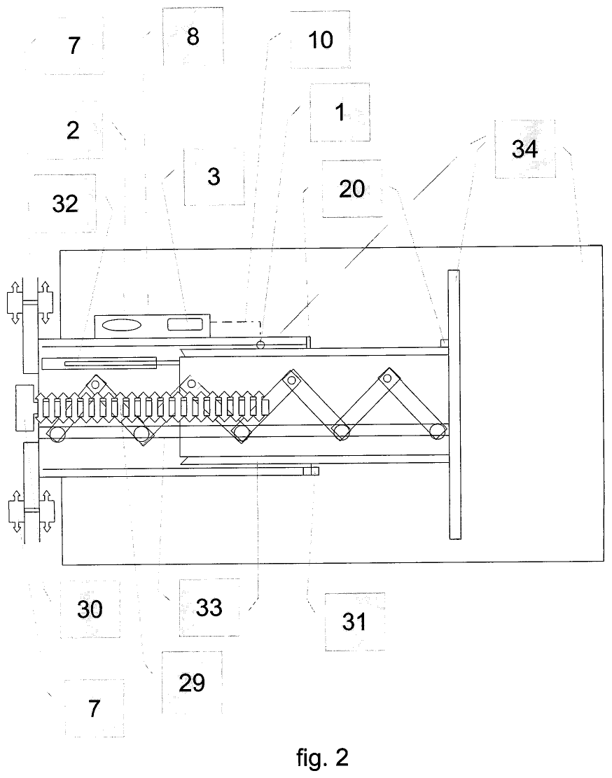

Another method to initiate activity is to worsen the preferred comfortable position of an inactive person and to urge the person to retake that previous preferred position. Lets take for example an elderly person laying at home. With an activated mobile flexible displacing device coupled to the bed (FIG. 2), the legs are pushed toward the head of the person. The device could even use sound to further urge the activity. The device could have one of the following preferred embodiment; The flexible displacing device that is coupled to the bed is comprising of a static and a moving component. The static component is coupled dismountable form to the bed structure, it is furnished with a flexible force producing equipment, containing a preloaded spring (29) with spring force adjusting mechanism (30). The spring is connected to the moving component applying controlled force. The moving component furnished with breaking mechanism (32) is guided in rails (33) between two end point movement buffers (20), it has the capability to move back and forth, and to apply force, to push the legs of the person. It is mechanically turned on and off. It has proper structure (24) for safety. Without the presence of counteracting force, assisted by the breaking mechanism (32) it moves in controlled manner to the end position. With the presence of counteracting force it can be pressed back to its beginning position. The scale of the acting forces can be predetermined by the spring force adjusting mechanism (30). Controlling is digital or mechanical, along the values of time, force level and repetition number. Signaling equipment can be implemented, to urge the person even more. Signaling equipment is connected to sensor (1) and it is activated, deactivated depending on the position of the moving component. The elderly person to retain its original preferred position has no other option but to straighten the legs, to push against the moving component. The applied controlled force and the movement limits of the device determines the level of activity (exercise power, length and repetition). Similar device with common functionality can be utilized for a sitting person as well. Placing and coupling the device under the table, the leg moving exercises can be performed for beneficial vital function improvements. Such exercises can be part of rehabilitation program offered by physiotherapist, executed at specialized institutes or at homes. Basically any part of the body can be trained by such activity enhancing devices with corresponding functionality, all needed is to couple a suitable device to any surrounding objects permanent or dismountable form.

One preferred embodiment could be of a table mounted arm and upper body activity initiating flexible displacing device is comprising of the forearm support ergonomic device for desk jobs by Ergorest Oy and flexible displacing device components. The arm structure of the Ergorest device is furnished with preloaded springs to produce flexible forces such way to create torque around its joints and movement on its arm structure. The Ergorest device is furnished furthermore with end point movement buffers, locking-unlocking subcomponent, further support element for the human arm and preloaded force adjusters for the springs. Thus creating a new device, that not only an ergonomic support of the forearm, but activity can be initiated through movement by flexible forces with controlled level, direction and period of time. To retain and withhold the movements, to keep arms and upper body in a suitable position for the required work at the desk, urges the user to use muscle forces, to be active, to do exercise.

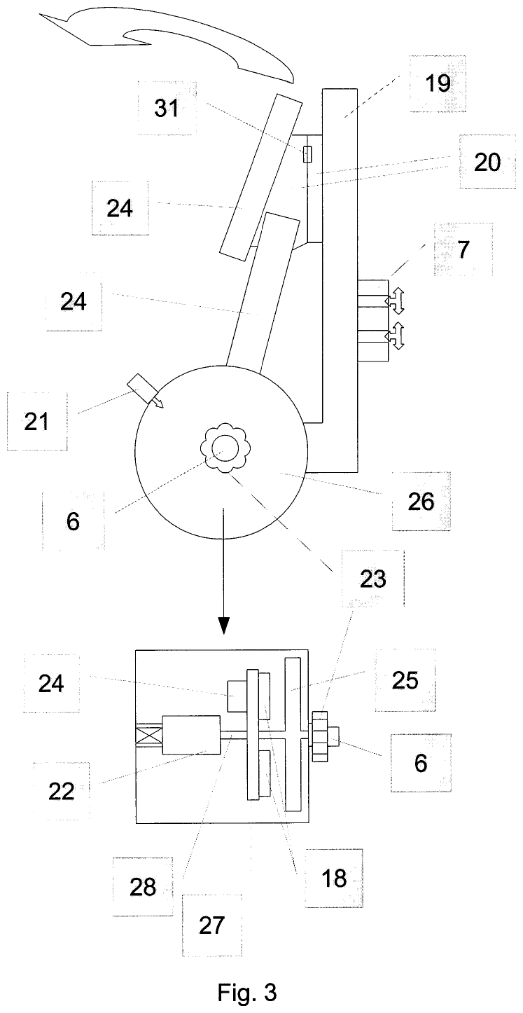

Electric motor (22) and magnetic plates (18) can also be applicable for flexible force producing equipment (FIG. 3), one of such preferred embodiment could be comprising a table mounted paddle flexible displacing device where the flexible force producing equipment is further comprising of a electric motor (22) and a magnetic resistance mechanism. The magnetic resistance mechanism has a magnetic break-load adjuster (23) for adjusting the scale of the acting flexible force. The paddle containing the exercise pad (24) is connected flexible way to the electric motor (22) by a free-turning wheel (27). The electric motor (22) is controlled by a on/off switch (6) and a distance limiter sensor. The electric motor (22) is furnished with a steel fly wheel (25). The flexible way of connection is comprising of the steel fly wheel (25), adjustable magnetic plates (18) and the free-turning wheel (27). By setting the distance by the magnetic break-load adjuster (23) between the fly wheel (25), the magnetic plates (18) and the free-turning wheel (27) the scale of the activated flexible force can by adjusted.

In case of standing position is necessary over lengthy periods, to increase activity at such circumstance, a suitable flexible displacing device would be placed under the feet or built into the floor. With capability of direction specific controlled flexible side movement, the standing person has to activate the muscles of the legs against the forces of the device, if nothing else, but for the sake of keeping a stable body position.

Nothing prevent pressure ulcer or decibus better than movement. During at physical conditions when people are confined to the bed or chair for long periods, life threatening pressure ulcer can develop. To prevent its development or help its healing, initiated activity could be solution. It can be stated that decibus develops due to prolonged inactivity. The skin receives continuous pressure, the local circulation is decreased to such level, that tissues are damaged and inflation becomes probable. To the consequence of activity the continuous pressure of the skin can be eased, the local circulation can be improved, not forgetting the favorable effect of movement to the total health condition of the body. The method can be as follows. By activated flexible displacing device certain parts of the body or the whole body is displaced and forced to take up new position. In the new body position the pressure zones are relocated to new arias of the skin. Initiated activity leads to the effort for movement, meaning the use of muscle force, that leads to improved internal vital body functions for better local and total well-being, thus for more improved opportunity for healing.

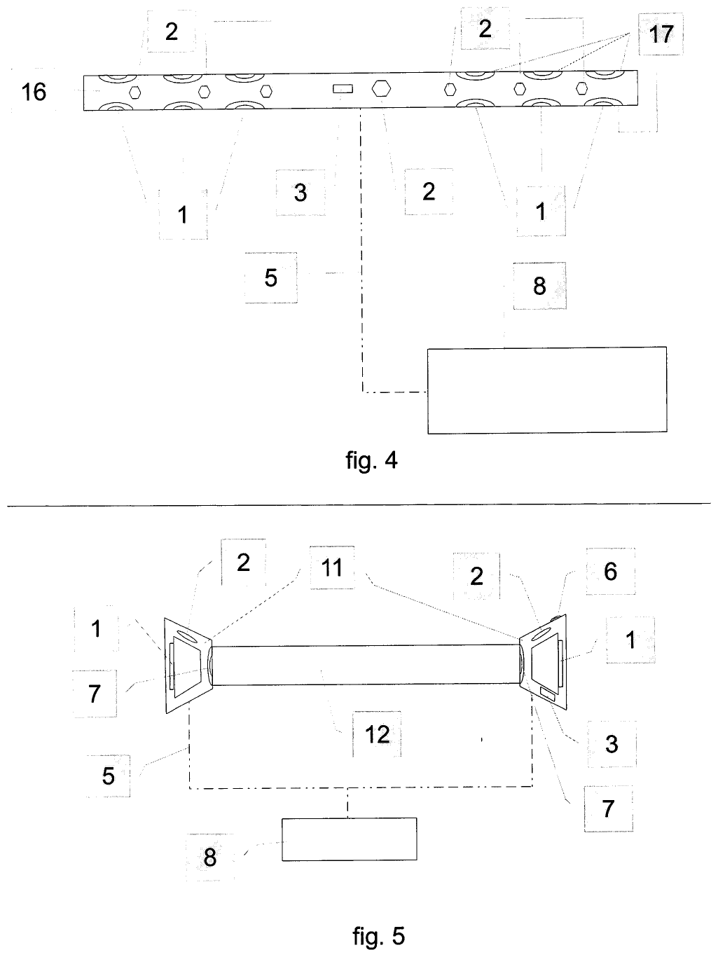

Several examples can be stated that in hospitals, rehabilitation environment, or at certain physical conditions how beneficial target oriented activity enhancing methods would be. The utilization of flexible force apparatus with signaling and measuring device can be significant due to their simplicity. Flexible force apparatus can be any article that is able to change its shape in a controlled flexible way due to the effect of outside force. Such example can be a flexible rod (FIG. 4), which can be flexible to bending or axial direction, with sensors (1) and signal sources outfitted on its surface. Muscle force is required for pulling or bending. Light signals (2) show on the surface of the rod where to grab and apply controlled force for bending or pulling. Light signal (2) and sound signal (3) shows the length of time for the required activity. Controller connects wireless form. Further example accordingly for one of the preferred embodiment is shown at (FIG. 5) with an elastic band. Another solution can be such device that is comprising of a mobile-phone and connected force sensors. The sensors can be placed onto any object and force can be applied to them with parts of the body. The mobile-phone through the sensors reads, by signals appearing on the display shows and controls the applied forces. Countless therapeutic and other devices can be developed in the future according to the method, which can increase the activity of any muscle group of the body, hence improving vital functions and the opportunity for life. Devices according to their configuration can also be utilized in the fitness market.

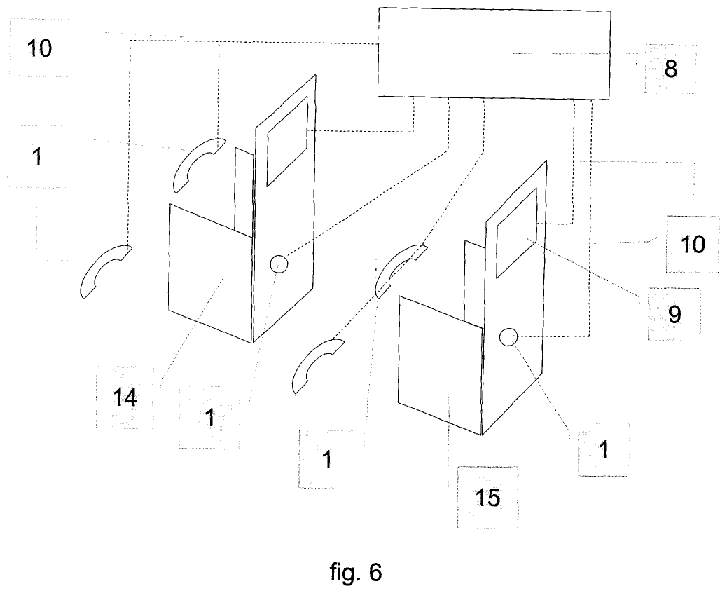

Method for reducing the harmful effects of physical inactivity in movie theaters can be shown as following (FIG. 6). Breaks are very uncommon these days at the movies, when people can leave or just have an opportunity to stand up. Therefor activity is an issue here as well. The length of today's movies are well over the time limit defined in medical research to be harmful being inactive, having accumulating effect. One of the preferred embodiment could be comprising of connected sensors (1) and signaling equipment. The signaling equipment is comprising of displays (9) placed on the back of each seat (15) and a conveniently located computer (8), that utilizes the data and runs the exercise routine. The displays (9) have wired (10) or wireless connection to the computer (8). The activity measuring sensors (1) can be placed on the ground around the seats (15) in specially formed spots, or they might have freely movable mobile form. In case of freely movable form, the sensors (1) are integrated part of a handheld appliance, which are stored in special holders at the seats. There are additional sensors (1) located in the seat (15) bottoms as well. The method can be as follows: sensors (1) evaluates weather the seat (15) is occupied, and measures the sitting weight of the person. Signaling equipment requests the sitting person to push the specifically placed sensors (1) in front of the seat (15) with left and right foot. Then the signaling equipment asks for the age and the body bight, that can be selected by pushing the sufficient sensors (1). The questions can be in visual form on the displays (9) or they can be projected on the movie screen prior to screening and be answered simultaneously by all attendees. From the available data the signaling equipment determines the required minimum force and length of time that are necessary for pushing the sensors (1) to turn off the on signal. According to the data, the signaling equipment determines that at what time, for how long and for how many repetitions will the signal be turned on for initiating activity. For urging purpose, at the beginning of activity cycle, signal begins to flash (preferably in a way that only the sitting person is effected) until the specific sensors (1) are pushed by each foot with controlled level of force and length of time. In case the push is stopped early or the level of force is insufficient, the flashing begins again for urging purposes to due again the exercise. The urging signal can not just be in the form of light, it can effect any senses of the person. Rational to initiate the activity together for everybody, to do the efforts simultaneously. Eventually the movies can be produced such a way that activity periods are purposely aligned to certain parts of the movie, where the structure of the movie and the urge of activity is coordinated.

The previously described activity initiating methods and respective devices combined completely or partially can work together affectively and possess countless possibilities.

Humans are made to be active. Activity is close related to our health, looks and self-satisfaction. Not only muscles need movement, movement is fundamental to cell metabolism, to the immune system, joints, circulation. It can be stated, activity is conducive to the complete system of organs of the body. For these reasons, it is extremely important to lessen the time of inactive periods and to help increase physical activity. The method and respective device according to this invention purposely offers solutions to this important subject that is not yet adequately in focus. For production the current state of technology is sufficient, all components are widely available. Quick and wide range of utilization is predicted.

### COMPONENT IDENTIFICATION LISTING

1 sensor
2 light signal
3 sound signal
4 signaling equipment coupled to nurse call-system console
5 wireless connection
6 on/off switch
7 dismountable coupling
8 controller
9 display
10 wire connection
11 handle integrated signaling and measuring device with outer controller
12 elastic band
13 hospital bed
14 nurse call-system console
15 row of chairs
16 flexible rod
17 grip position
18 magnet plate
19 table mounted flexible displacing device structure
20 end point movement buffer
21 distance limiter sensor to stop electric motor
22 electric motor
23 magnetic break-load adjuster
24 exercise pad and pedal structure
25 steel flywheel
26 flexible force producing equipment housing
27 free-turning wheel
28 shaft
29 preloaded-spring
30 spring force adjusting mechanism
31 locking/unlocking apparatus
32 breaking mechanism
33 rail
34 displacing pad and structure for in-bed use

1. For the aim to reduce the harmful effects on health during the circumstances of physical inactivity, method to initiate and control physical activity by suitable signaling and measuring device, wherein one or more signaling equipment is initiating physical activity by one or more controlled signal and is measuring muscle forces by one or more sensor, wherein the signal is initiating predetermined parts of the human body to act onto the sensor with physical forces of controlled scale, duration and repetition, wherein the muscle contractions are isometric or none-isometric or both, wherein the signaling equipment is evaluating the measured values of the sensor and is controlling the signal accordingly, wherein parts of the signaling and measuring device is coupled as permanent supplement or as a dismountable attachment to other conventional or customary objects or articles, such as bed, table, wall, cabinet, draw, keyboard holder, nurse call-system console and the like, wherein software is controlling the operation, that is running internally or on connected device or through the Internet or any of their combination.

2. For the aim to reduce the harmful effects on health during the circumstances of physical inactivity, method to initiate and control physical activity by suitable flexible displacing device, wherein one or more activated flexible force producing equipment providing flexible displacement is displacing predetermined parts of the human body in a controlled manner, such way that the acting forces have controlled path, direction, size, beginning, duration and repetition and the parts of the body effected can retain or move against those displacing forces, wherein the activated flexible displacing device is making the preferred comfortable body position of the person worse, such way, that the person is urged to retake a body position against the acting flexible forces or the person is urged to turn off an on signal by retaining, moving against the activated flexible forces or both, wherein the form of the signal is effecting the human senses, such as light or sound or smell or vibration or touch or sensation or projection on display or the nature of the signal is such, that it is desirable turning off or any of their combination.

3. Method of claim 2, wherein software is controlling the operation, that is running internally or on connected device or through the Internet or any of their combination.

4. Method of claim 1, wherein the form of the signal is effecting the human senses, such as light or sound or smell or vibration or touch or sensation or projection on display or the nature of the signal is such, that it is desirable turning off or any of their combination.

5. Method of claim 2, wherein motor, weight, spring, pneumatic or hydraulic piston, magnetic force driven apparatus or any of their combinations is producing the required forces.

6. Method of claim 1, wherein to reduce the ill effects of inactive periods or excessive laying periods in beds at healthcare, rehabilitation, elderly care centers or at home, to increase muscle activity, joint movement, inner organ function, such as circulation, immune system, or the like, signaling and measuring device or flexible displacing device or both is initiating controlled muscle work by having effect direct or indirect mode on predetermined parts of the human body.

7. Method of claim 1, wherein at physical conditions to be confined to bed or chair, to reduce the development and harmful effect of the life threatening pressure ulcer, also known as Decibus, signaling and measuring device or flexible displacing device or both is urging the inactive person to move in the bed or chair and to change to a new body position.

8. Method of claim 2, wherein that is applying in such way, that is limiting to such extent, that its process is adapted to the circumstances, to the environment in which it occurs and preferably it is unobtrusive

9. Signaling and measuring device for initiating physical activity, or for therapeutic, or for rehabilitation purpose particularly adapted for carrying out method according to claim 1 comprising of one or more signaling equipment capable to produce controlled signal, and one or more sensor, and one or more data transmission capable connection, wherein elements of the device, which are in contact with the body parts of the person that are applying muscle forces, are connected direct or indirect mode to the sensor, wherein elements of the device are integrated or coupled as a supplement permanently or dismountable to other conventional or customary objects or appliances, wherein the signaling equipment is comprising of one or more signal source and one or more controller, wherein the signaling equipment has digital or analog or mechanical operation or any of their combination, wherein the signaling equipment has stand-alone operation or has the capability to receive control from outer source, wherein the produced signal effects one or more human senses, such as light, sound, smell, vibration, form of sensations through the body, display projection or the like, or any of their combination.

10. Flexible displacing device for initiating physical activity or for healthcare purpose or for rehabilitation purpose particularly adapted for carrying out method according to claim 2, wherein the part of the device that is in contact with the person has direct or indirect link to flexible force producing equipment, wherein the activation, deactivation, scale, duration and repetition of the produced force are controlled by one or more controller, wherein the movement range of the device is governed between endpoints along predetermined path, wherein the controller has mechanical or analog or digital operation or any of their combinations, wherein the physical activity is initiated by displacement or there is connected signaling equipment, sensor or both.

11. Device of claim 10. wherein the force is generated in the flexible force producing equipment by motor, such as electric, magnetic, linear, rotational, or the like or spring such as spiral, preloaded, torsional, rubber ring, or the like, or piston, such as hydraulic, pneumatic, or the like, or magnets, or interaction of magnets, or weight or any of their combination.

12. Device of claim 10, wherein it is coupled to wheelchair in a dismountable form or it is an integrated part of a wheelchair.

13. Device of claim 9. wherein the signaling equipment is a mobile smart device, which mobile smart device is connected to one or more sensor by wired or wireless connection, wherein the sensor is integrated part of a holdable, handheld appliance.

14. Device of claim 10, wherein it has mobile, portable form with the capability to be coupled or attached to other objects, articles such as bad, table, wheelchair, cabinet, draw, and the like or it has a permanent form, wherein it is manufactured or supplied together as an integrated part of other objects or it is an addition to other objects as a built-on supplement

15. Device of claim 9, wherein the sensors are located on rows of chairs or on separate chair structures or at other reachable location around the chair or the sensors have hand held freely movable component form, wherein the sensors have wired or wireless connection to the signaling equipment, wherein the controlled signal is realized on mobile smart device or on the structure of the chairs, or on available displays or through earpieces, wherein the control is originating from a central controlling appliance or from the appliance that supplies the signal or both, at transportation or at such locations of buildings where there are rows of chairs.

16. Device of claim 9, wherein the signaling equipment and sensors are coupled to a flexible force apparatus, wherein the flexible force apparatus is any article that is able to change its shape in a controlled flexible way due to the effect of outside force, such as a flexible rod or a flexible band, or the like.

17. Device of claim 10, wherein the flexible displacing device is coupled direct or indirect mode to the structure of the forearm support ergonomic device for desk jobs, in such a way that the force creates torque around the joints and movement on the arm structure.
